(19) United States
(12) Patent Application Publication
(10) Pub. No.: US 2005/0089556 A1
(43) Pub. Date: Apr. 28, 2005

(54) Composition for treating hemorrhoids

(76) Inventor: L. Gaye Carroll, Magnolia, AR (US)

Correspondence Address: R. Keith Harrison, 2139 E. Bert Kouns, Shreveport, LA, 71105, US

(21) Appl. No.: 10/424,394

(22) Filed: Apr. 28, 2003

Related U.S. Application Data
(63) Continuation-in-part of application No. 10/190,718, filed on Oct. 10, 2002.

(51) Int. Cl. A61K 9/64

(52) U.S. Cl. 424/456

(57) ABSTRACT

A hemorrhoid-treating composition which includes a mixture of the antiseptic and bactericide gentian violet (methylrosaniline) and a lubricant such as petroleum jelly, also known as petrolatum or mineral jelly. In another embodiment, the composition is prepared as a suppository using a suitable suppository base. The composition imparts a soothing and healing effect on swollen hemorrhoids, and particularly, on bleeding hemorrhoids.

### CROSS-REFERENCE TO RELATED APPLICATIONS

This application is a continuation-in-part of application Ser. No. 10/190,718, filed Oct. 10, 2002.

### FIELD OF THE INVENTION

The present invention relates to a composition which is particularly useful in the treatment of hemorrhoids. More particularly, the present invention relates to a hemorrhoid-treating composition which includes an ointment mixture of the antiseptic and bactericide gentian violet (methylrosaniline) and a lubricant such as petroleum jelly, also known as petrolatum or mineral jelly. The composition further includes a suppository capsule. The composition imparts a soothing and healing effect on swollen hemorrhoids, and particularly, on bleeding hemorrhoids.

### BACKGROUND OF THE INVENTION

Hemorrhoids are swollen blood vessels in the rectum. There are two basic types of hemorrhoids: internal and external. Internal hemorrhoids are swollen and inflamed veins far up in the rectum. Internal hemorrhoids cannot be seen or felt and usually are not painful due to the paucity in nerve endings in the upper portion of the rectum. While internal hemorrhoids are most commonly manifested by anal bleeding, they may prolapse, or protrude outside the anal sphincter. Usually, prolapsed internal hemorrhoids may be gently pushed back into place in the rectum.

External hemorrhoids are swollen blood vessels in the anus and are usually manifested by pain as well as bleeding. When external hemorrhoids prolapse, or protrude from the anal sphincter, blood clots sometimes form, causing an extremely painful condition known as thrombosis. While they usually disappear by themselves within about a week, thrombosed hemorrhoids may be removed by a physician or may be treated with a pain-reducing medication to reduce the pain.

It is believed that hemorrhoids are caused by the exertion of abdominal pressure on rectal veins, causing the veins to swell and become irritated. The abdominal pressure may be caused by a variety of factors and conditions including obesity, pregnancy, prolonged standing or sitting, liver disease, straining during bowel movements, coughing, sneezing, vomiting or holding the breath during physical activity. Hemorrhoids are largely preventable by the adoption of a high-fiber diet. On the other hand, persons whose diet consists largely of low-fiber, processed foods tend to run the highest risk of developing hemorrhoids. Furthermore, inadequate fluid intake can contribute to the development of hemorrhoids by causing the development of hard stools which irritate and inflame the rectal veins.

About half of persons living in the United States will be afflicted with hemorrhoids at some point during their lives. Hemorrhoids most often strike persons between the ages of 20 and 50. Some evidence indicates that “weak” veins, which are most susceptible to developing hemorrhoids, are inherited.

### SUMMARY OF THE INVENTION

The present invention is generally directed to a composition for the treatment of hemorrhoids. More particularly, the present invention relates to a hemorrhoid-treating composition which includes a mixture of the antiseptic and bactericide gentian violet (methylrosaniline) and a lubricant such as petroleum jelly, also known as petrolatum or mineral jelly. In another embodiment, the composition is prepared as a suppository using a suitable suppository base. The composition imparts a soothing and healing effect on swollen hemorrhoids, and particularly, on bleeding hemorrhoids.

### DESCRIPTION OF THE PREFERRED EMBODIMENTS

The present invention includes a composition for the treatment of hemorrhoids, particularly external hemorrhoids. The composition is easy to apply and promotes pain reduction, as well as healing, of the hemorrhoids, including inflammation and torn tissue associated with hemorrhoids. In a preferred embodiment, the composition includes an ointment mixture of non-prescription strength, 1% gentian violet (methylrosaniline) which is thoroughly mixed with a lubricant such as petroleum jelly, also known as petrolatum or mineral jelly. The methylrosaniline is an effective antiseptic and bactericide which prevents or fights infection and reduces pain. The lubricant provides a vehicle for effective delivery of the methylrosaniline to the inflamed hemorrhoid tissue. In a typical embodiment, the composition includes about seven (7) drops of the liquid methylrosaniline bactericide/antiseptic per teaspoon of the lubricant. The bactericide/antiseptic may be dispensed from a medicine dropper such as an EZY CARE (trademark) straight-tip or bent-tip, glass medicine dropper available from Apothecary Products, Inc. of Minneapolis, Minn. The EZY CARE (trademark) medicine dropper has a capacity of 1 ml of liquid, or approximately 30-35 drops. Accordingly, in a preferred embodiment the composition mixture includes at least about 0.2 ml to about 0.3 ml of the methylrosaniline per teaspoon of lubricant. However, it is understood that other types of medicine droppers may be used to dispense the drops into the lubricant. After the methylrosaniline is added to the lubricant, the two components are thoroughly mixed until the composition mixture assumes the substantially homogenous violet color of the methylrosaniline. After formation, the composition is typically placed in a typically 6 oz. capped, plastic applicator tube for subsequent application.

In another embodiment, the composition is prepared in the form of a suppository capsule using a suitable suppository vehicle or base. Preferably, the suppository base is glycerinated gelatin, although alternative suppository bases, including cocoa butter and polyethylene glycol, in non-exclusive particular, may be used. The active ingredient bactericide/antiseptic gentian violet (methylrosaniline) is added to the suppository base in a quantity of typically at least about 0.2 ml to about 0.3 ml of 1% non-prescription strength, gentian violet (methylrosaniline) per teaspoon of suppository base. The methylrosaniline may be dispensed from a medicine dropper such as an EZY CARE (trademark) straight-tip or bent-tip, glass medicine dropper available from Apothecary Products, Inc. of Minneapolis, Minn., for example. The therapeutic quantity of methylrosaniline equates to typically at least about seven (7) drops of the methylrosaniline per teaspoon of suppository base. The methylrosaniline is then thoroughly mixed with the suppository base to obtain a substantially homogenous suppository mixture. One teaspoon of the suppository mixture may be formed or shaped into two suppository capsules each having dimensions of typically about 0.25 inch in diameter and 1.5 inch in length, with a tapered end for anal insertion of the suppository. In that case, each suppository capsule typically has at least about 2-4 drops of the methylrosaniline. However, it is understood that the suppository mixture may be shaped into suppository capsules having any suitable dimensions and shape.

One possible method of applying the ointment composition to the inflamed hemorrhoidal tissue includes dispensing the composition from a typically 6-ounce, capped plastic tube. A replaceable applicator cap having a smooth, rounded applicator tip may be provided on the capped tube for applying the composition to the hemorrhoids in the anal area. Preferably, the applicator tip is ¾″ long, and the top of the 6-ounce tube, as well as the base of the applicator tip, is ¼″ in diameter. The applicator tip tapers from the ¼″ base to the ⅛″ tip thereof. Applicator openings are provided in the sides of the applicator tip for dispensing the composition from the tip to the inflamed hemorrhoidal tissue.

It is understood that the tube and applicator cap heretofore described represent only one example of storage and application of the composition. Accordingly, the composition may be stored in any type of suitable container for subsequent application, and may be applied through the applicator cap heretofore described or through any other type of applicator or other instrument suitable for the purpose. The composition may also be applied manually to the inflamed area. Furthermore, the fingers of the patient afflicted with prolapsed hemorrhoids may lubricate his or her fingers with the composition mixture and use the lubricated fingers to gently push the prolapsed hemorrhoidal tissue back into the anal canal. The composition mixture reduces hemorrhoidal pain and promotes healing of the inflamed hemorrhoidal tissue.

The invention will be better understood by consideration of the following examples.

### EXAMPLE 1

A composition mixture was prepared by dropping seven (7) drops of non-prescription strength gentian violet (methylrosanaline) bactericide/antiseptic, using an EZY CARE (trademark) medicine dropper, into one (1) teaspoon of petroleum jelly and then thorougly mixing the methylrosanaline with the petroleum jelly until the resulting composition mixture assumed the substantially homogenous violet color of the methylrosanaline. The composition mixture was placed in 6-ounce capped tube having an applicator cap for subsequent application of the composition to inflamed hemorrhoidal tissue.

### EXAMPLE 2

The composition mixture prepared according to EXAMPLE 1 above was applied to the inflamed hemorrhoidal tissue of a patient using the applicator cap on the tube. The composition mixture was applied as often as necessary to relieve pain. The composition mixture significantly reduced pain associated with the hemorrhoids and promoted healing of the inflamed hemorrhoidal tissue.

### EXAMPLE 3

A patient afflicted with inflamed hemorrhoids applied the composition mixture prepared according to EXAMPLE 1 to the patient's inflamed hemorrhoidal tissue using the patient's fingers. The composition mixture was applied as often as necessary to relieve pain. The composition mixture significantly reduced pain associated with the hemorrhoids and promoted healing of the inflamed hemorrhoidal tissue.

### EXAMPLE 4

A patient afflicted with prolapsed hemorrhoids lubricated the patient's fingers using the composition mixture prepared according to EXAMPLE 1 above. The patient used the lubricated fingers to gently push the prolapsed hemorrhoids back into the patient's anal canal. The composition significantly reduced pain associated with the hemorrhoids and promoted healing of the inflamed hemorrhoidal tissue.

### EXAMPLE 5

Suppository capsules were prepared by dropping seven (7) drops of non-prescription strength gentian violet (methylrosanaline) bactericide/antiseptic, using an EZY CARE (trademark) medicine dropper, into one (1) teaspoon of glycerinated gelatin and then thorougly mixing the methylrosanaline with the glycerinated gelatin until the resulting suppository mixture was substantially homogenous. The suppository mixture was shaped into two (2) suppository capsules for subsequent anal insertion to treat inflamed hemorrhoidal tissue.

### EXAMPLE 6

A suppository capsule prepared according to EXAMPLE 5 above was applied by anal insertion to inflamed hemorrhoidal tissue of a patient. The suppository capsules were applied as often as necessary to relieve pain. The suppository capsules significantly reduced pain associated with the hemorrhoids and promoted healing of the inflamed hemorrhoidal tissue.

While the preferred embodiments of the invention have been described above, it will be recognized and understood that various modifications can be made in the invention and the appended claims are intended to cover all such modifications which may fall within the spirit and scope of the invention.

1. A suppository capsule comprising:
a pharmaceutically acceptable suppository base; and
a therapeutic quantity of methylrosanaline mixed with said suppository base.

2. The suppository capsule of claim 1 wherein said suppository base comprises glycerinated gelatin.

3. The suppository capsule of claim 1 wherein said methylrosanaline is present in said suppository base in a ratio of at least about 0.2 ml to about 0.3 ml of said methylrosanaline per teaspoon of said suppository base.

4. The composition of claim 3 wherein said suppository base comprises glycerinated gelatin.

5. A suppository capsule prepared according to a process comprising:
preparing a suppository mixture by mixing a therapeutic quantity of methylrosanaline with a pharmaceutically acceptable suppository base; and
defining said suppository capsule by shaping said suppository mixture.

6. The composition of claim 5 wherein said suppository base comprises glycerinated gelatin.

7. The composition of claim 5 wherein said methylrosanaline is present in said suppository mixture in a ratio of at least about 0.2 ml to about 0.3 ml of said methylrosanaline per teaspoon of said suppository base.

8. The composition of claim 7 wherein said suppository base comprises glycerinated gelatin.

9. A suppository capsule for treating hemorrhoids, comprising:
a pharmaceutically acceptable suppository base; and
a suppository mixture prepared by mixing a therapeutic quantity of methylrosanaline with said suppository base,
wherein said suppository mixture is shaped to define said suppository capsule.

10. The method of claim 9 wherein said suppository base comprises glycerinated gelatin.

11. The method of claim 9 wherein said methylrosanaline is present in said suppository mixture in a ratio of at least about 0.2 ml to about 0.3 ml of said methylrosanaline per teaspoon of said suppository base.

12. The method of claim 11 wherein said suppository base comprises glycerinated gelatin.
